(19) United States
(12) Patent Application Publication
(10) Pub. No.: US 2025/0342949 A1
(43) Pub. Date: Nov. 6, 2025

(54) DEVICE AND METHOD FOR INTERACTIVELY PERFORMING SAMPLING AND/OR DISINFECTION

(71) Applicants: FRESENIUS MEDICAL CARE DEUTSCHLAND GMBH, Bad Homburg (DE); FRESENIUS MEDICAL CARE MAGYARORSZÁG EGÉSZSÉGÜGYI KFT., Budapest (HU); FRESENIUS MEDICAL CARE GMBH, Bad Homburg (DE)

(72) Inventors: Thiemo DALDRUP, Bad Homburg (DE); Christopher HENNEKEN, Bad Homburg (DE); Michael CHRIST, Bad Homburg (DE); Thomas POHL, Bad Homburg (DE); Alexander WAGNER, Bad Homburg (DE); Ferenc VERES, Bad Homburg (DE)

(73) Assignees: FRESENIUS MEDICAL CARE DEUTSCHLAND GMBH, Bad Homburg (DE); FRESENIUS MEDICAL CARE MAGYARORSZÁG EGÉSZSÉGÜGYI KFT., Budapest (HU); FRESENIUS MEDICAL CARE GMBH, Bad Homburg (DE)

(21) Appl. No.: 18/876,203

(22) PCT Filed: Jun. 16, 2023
(86) PCT No.: PCT/EP2023/066235

§ 371 Date: Dec. 18, 2024

(30) Foreign Application Priority Data
Jun. 24, 2022 (DE) 10 2022 115 846.2

(51) Int. Cl. G16H 40/40; G16H 40/63

(52) U.S. Cl. CPC G16H 40/40; G16H 40/63

(57) ABSTRACT

The present invention relates to a device for interactively carrying out sampling and/or disinfection of a system, more particularly a water technology system, wherein the device has a user interface and a controlling unit which is programmed to identify the system, to load, from a database, historical data relating to at least one operating process of the system and to automatically create, on the basis of the historical data, a execution sequence for future sampling and/or disinfection of the system, wherein the execution sequence contains at least one specific task for a user, which task is communicated to the user at at least one predefined time, wherein the user is asked, by means of the user interface, to enter a feedback regarding the at least one specific task and/or the completion thereof by the user, following which the controlling unit independently adjusts and/or updates the execution sequence for sampling and/or disinfection on the basis of the feedback of the user.

The present invention relates to a device and a method for interactively carrying out sampling and/or disinfection of a system, more particularly in the field of medical technology and or water technology in the medical context, for example for the creation of dialysis water, ultra-pure water etc.

Particularly stringent quality standards apply for the creation of water for the medical field in order to ensure appropriate patient safety at all times. The activities of several actors, for example the hospital staff, service engineers for a water technology system and the staff at external laboratories who analyze the samples, must be coordinated.

In the often hectic day-to-day work of the hospital, the planning and carrying out of sampling and/or disinfection of water technology systems is therefore very time-consuming.

Against this background, the present invention addresses the problem of reducing or completely overcoming the disadvantages of the prior art. More particularly, the present invention addresses the problem of how to make the sampling and/or disinfection of a system faster and more user-friendly.

This object is achieved by the subject matters of the independent claims. Developments of the invention are the subject-matter of the dependent claims.

The invention proposes a device for interactively carrying out sampling and/or disinfection of a system, more particularly a water technology system, or at least one part thereof, wherein the device has a user interface and a controlling unit which is programmed
to identify the system or the at least one part thereof,
to load, from a database, historical data relating to at least one operating process of the system or the at least one part thereof, and
on the basis of the historical data, to automatically create an execution sequence for future sampling and/or disinfection of the system or the part thereof, wherein the execution sequence contains at least one specific task for a user, which is communicated to the user at at least one predefined point in time, wherein
the user is requested, by means of the user interface, to input a feedback regarding the at least one specific task and/or the completion thereof by the user, following which the controlling unit independently adjusts and/or updates the execution sequence for sampling and/or disinfection on the basis of the feedback of the user.

A device according to the invention preferably does not have a write function in relation to settings and/or control units of the system or a part thereof. A device according to the invention preferably operates in a read-only manner in relation to settings and/or control units of the system or a part thereof.

Alternatively or in addition to the feedback by the user, a feedback regarding the at least one specific task and/or the completion thereof by the user can be provided to the device by the system and/or the at least one part thereof, for example a reverse osmosis system.

The fact that the controlling device obtains a feedback can mean, for example, that in the event of a disinfection of a reverse osmosis system, information from same about the disinfection process is transmitted to the device by means of digital communication.

The feedback can comprise time stamp, status information and/or measurement values. However, the feedback can also be a human input, for example if automated capture was not possible.

In other words, a device according to the invention preferably provides an automated and interactive tool which supports a user during the creation of an execution sequence for sampling and/or disinfection of a system, which tool independently captures relevant data and interactively supports the user during the carrying out of sampling and/or disinfection of the system and/or guides the user through the process. The present invention can therefore be understood to interactively support a user in an interactive manner during the operation of a system for the sampling and/or disinfection of the system.

The expression “device for interactively carrying out sampling and/or disinfection” is therefore not necessarily understood to mean that the device carries out sampling and/or disinfection itself or alone, but can also be interpreted to mean that the device interacts with a user in order for sampling and/or disinfection to be carried out interactively with the user.

For example, the device can output specific action recommendations and/or tasks to the user during an execution sequence, monitor the carrying out of the execution sequence and on the basis thereof adjust or update the sequence of action steps which are to be carried out by the user.

However, it is also in principle conceivable that the device carries out the execution sequence itself, so for example carries out the sampling processes or disinfection processes itself and interacts with the user only during the creation of the execution sequence.

Sampling and/or disinfection can comprise one or more sampling and/or disinfection processes.

An execution sequence created within the framework of the present invention can contain tasks or action instructions which are to be carried out by a user in a freely definable time period.

For example, the execution sequence can comprise a yearly plan, weekly plan or daily plan for the sampling and/or disinfection processes to be carried out on a system, preferably including their time periods and procedures.

For example, a device according to the invention preferably interacts with the user in that the device generates specific tasks relating to carrying out the sampling and/or disinfection of the system for the user and the controlling unit independently adjusts and/or updates the execution sequence for sampling and/or disinfection on the basis of the feedback of the user.

For example, the task for the user can consist in visually inspecting a valve for taking a sample. After the task has been completed, the user can for example provide feedback that the valve is OK, as a result of which the device adheres to the created execution sequence. The execution sequence preferably contains specific information regarding the time, place and/or type of sampling and/or disinfection.

Alternatively, the user can for example provide feedback that the valve is not OK, as a result of which the device independently updates the execution sequence, for example using available service engineer appointments (based on the created execution sequence, the sampling is planned for the morning, but the engineer does not have time until the day after tomorrow, so the execution sequence is adjusted so that the sampling takes place the day after tomorrow).

The execution sequence is preferably created not exclusively on the basis of historical data and these data merely constitute one parameter which is considered.

Other parameters, such as a type of system, a location of the system, regulatory provisions applicable at this location and/or the operating procedures common at this location, as well as other parameters, can also be considered.

It is also conceivable that no historical data are considered during the creation of the execution sequence, and instead the execution sequence is created on the basis of user inputs.

After an adjustment and/or update of the execution sequence, the device preferably prompts the user to approve or confirm the execution sequence. Here, the user can preferably edit individual parts of the execution sequence, for example change a time or procedure of a planned sampling process. The user can preferably approve or confirm the entire execution sequence or partial sections thereof, for example for a particular calendar week or a month.

A system preferably comprises at least one of the following parts: a water pre-treatment system having at least one physical filter, a softener and an active carbon filter; a reverse osmosis system; sensors and a monitoring apparatus; one or more gateways which transmit data from one of the aforementioned parts to a server or a cloud; a ring line for transporting water, to which ring line medical apparatuses, more particularly dialysis machines, can be connected.

The system can preferably be disinfected with hot water and has defined withdrawal points for samples for checking the microbiological quality of the water.

Alternatively to disinfection with hot water, the system is designed for chemical disinfection. In the case of chemical disinfection, it is not heat which kills the pathogens but rather aggressive chemicals such as, for example, a mixture of 25-50% hydrogen peroxide, 2.5-5% peracetic acid and 2.5-5% acetic acid. Alternatively, the system is designed for disinfection with both hot water and chemical disinfection. A disinfection process can thus be carried out thermally or chemically.

According to an embodiment, the controlling unit is further programmed to prompt a user, by means of the user interface, to confirm a created execution sequence for future sampling and/or disinfection of the system or the part thereof.

The controlling unit can preferably independently determine whether a single user or a plurality of users, for example two users, are required for the confirmation or approval. The determination takes place for example on the basis of a criticality of a planned process, the number of people involved in a planned process or another relevant parameter.

The controlling unit can be programmed to trigger, after confirmation has been provided, an output unit to output at least one label, on which preferably a sampling place, a system, or a part thereof, to be sampled, a type of sample, a time of sample-taking, an instruction to examine the sample and/or a name of the person carrying out the sampling is specified.

For example, the controlling unit can actuate a printer to create a label for a sample container.

Alternatively or additionally, a dispatch note or an executional excerpt or an action instruction can also be output. For example, for a confirmed planned disinfection, an executional excerpt in the form of a specific action instruction can be output, containing a time of disinfection, a disinfection process (e.g. “schema A”) and operating instructions for the device to be disinfected (e.g. “on the reverse osmosis system, first press switch B, then wait etc.”).

Data within the meaning of the present application can preferably be digital data.

According to an embodiment, the controlling unit is programmed to capture a location of the system or the at least one part thereof, to load the local circumstances currently applicable at this location, more particularly regulatory requirements, public holidays, responsibilities, specified processing intervals and/or operating processes and to take account of the local circumstances during creation of the execution sequence for future sampling and/or disinfection of the system or the part thereof.

For example, the fact that a local public holiday applies in a country in which a particular system is set up is captured. Since samples are not processed on this day in this country, the device takes account of the public holiday, and associated storage times of a taken sample, when creating the execution sequence. To do this, the device preferably independently accesses data from databases without requiring active input from a user.

Regulatory requirements can also be captured automatically. For example, only process schemas A, B and C are permitted for disinfection in a first country, but process schemas C, D and E are permitted for disinfection in a second country. During creation of an execution sequence, therefore, it is preferably automatically considered which process schemas are specified or permitted in the country of the system or the part thereof.

This can for example improve the clarity during interaction of the device with a user, since the user is for example offered tailored selection lists (e.g. in the form of dropdown menus).

If the fact that a system for which sampling and disinfection is to be carried out interactively by a user in interaction with a device according to the invention is located in a first country was captured, the user is, during interactive creation of the execution sequence, offered for selection a list in the form of a dropdown menu containing only the process schemas A, B and C defined as permitted based on the location of the system, for example. This increases the user-friendliness and efficiency of the communication of the device with the user, because the user need only make a selection from the preselected permitted process schemas and is not distracted by irrelevant parts such as the impermissible process schemas D and E, for example, as would be the case with a conventional list containing parts A, B, C, D and E.

In other words, the device, more particularly the controlling unit, independently carries out a preselection of parts of at least one selection list offered to the user during the creation of an execution sequence in interaction with the device.

The selection lists offered to the user by the device are thus preferably not static, but dynamic, since the parts of each list change depending on the captured user inputs or other captured data, for example regarding the type or location of a system.

According to an embodiment, the controlling unit is thus programmed to automatically create at least one dynamic selection list on the basis of the identified system or the part thereof and preferably an associated location, which selection list preferably enables the user to make a selection regarding the system, at least one part thereof, a planned disinfection method, a planned sampling, a place, preferably of the sample-taking, a type of planned sampling and/or an examination of samples, wherein the parts of the list are automatically adjusted in response to input made by the user by means of the user interface and/or data independently captured by the device.

A dynamic list is preferably understood to mean that the parts of the list are not static but are adjusted by the device automatically in response to input made by the user by means of the user interface and/or data captured independently by the device. In other words, a preselection of parts of the preferably takes place by the device instead of on the basis of an evaluation of relevance and/or a plausibility check by the control unit.

For example, Spain was captured as the location of a first system, and Germany was captured as the location of a second system. Different legal requirements for the operation of medical water technology systems apply, for example, in the two countries. Thus examinations X and Y of a microbiological sample are required in Spain, while examinations Y and Z of a microbiological sample are required in Germany, for example.

Instead of offering the user a list containing parts X, Y and Z for selection of a planned examination of the sample in both cases, a device according to the invention restricts the list preferably in advance to relevant parts. For example, if the system was identified as being located in Spain, the user sees a list containing parts X and Y. This reduces the amount of irrelevant information from which the user has to find the relevant options.

According to an embodiment, the controlling unit is further programmed to prompt a user, by means of the user interface, to confirm a created execution sequence for future sampling and/or disinfection of the system or the part thereof, for which by means of the user interface the created execution sequence is output in the form of an overview, which enables the user to confirm the execution sequence to be confirmed in a centralized manner with just one action or to reject the confirmation.

Preferably, the user does not necessarily have to actively make an input for each part of the execution sequence, for example does not individually confirm each individual sampling process or disinfection process including associated date, time, process schema etc., and can instead confirm the execution sequence in a centralized manner with preferably just one action, for example a click, for example can approve, in a centralized manner, an annual plan with all the sampling processes and/or disinfection processes planned for that year.

This significantly saves t time, because the user does not have to create an execution sequence taking account of numerous boundary conditions, and instead merely has to preferably approve this and, in the event of approval, does not have to approve each individual item.

According to an embodiment, the controlling unit is further programmed to create an execution sequence for future sampling and disinfection of the system or the part thereof, wherein after each disinfection, sampling is planned, by means of which sampling the effectiveness of the disinfection is validated.

The individual processes in the disinfection, more particularly the heat disinfection or chemical disinfection, are preferably planned by a device according to the invention jointly and in a coordinated manner with the processes for sampling by means of which the success or effectiveness of the disinfection are checked. The device according to the invention accompanies and supports a user preferably during creation of an execution sequence for disinfection and/or sampling and during the carrying out of disinfection and/or sampling, for example by outputting specific tasks.

According to an embodiment, the controlling unit is programmed to provide the user, by means of the interface, with an option of editing a created execution sequence. For example, a user can first refuse to confirm a created execution sequence, can edit same and then confirm the edited execution sequence.

According to an embodiment, the controlling unit is programmed to capture data regarding at least one sampling and/or disinfection of the system, or the at least one part thereof, which has actually taken place, and to compare said data with data regarding at least one associated sampling and/or disinfection of the system, or the at least one part thereof, planned as part of the execution sequence.

For example, an overview of events planned as part of the execution sequence and events which actually occurred or did not occur can be displayed to the user. The events which were planned and the events which actually occurred are preferably compared with one another in an integrated overview. The controlling unit can use an automatic assignment mechanism here which automatically assigns an event which actually occurred to an associated planned event.

According to an embodiment, the controlling unit has an automatic assignment mechanism which is programmed to capture captured data regarding at least one sampling and/or disinfection of the system, or the at least one part thereof, which has actually taken place and to assign said data to associated data regarding at least one corresponding planned sampling and/or disinfection of the system or the at least one part thereof, and the controlling unit preferably carries out a plausibility check before the assignment.

The result of the comparison can be displayed to the user by means of an output and/or the controlling unit can be designed to independently adjust and/or update the created plan for future sampling and/or disinfection on the basis of the captured data and/or the result of the comparison.

The result of the comparison can relate to an overall event, for example a heat disinfection was planned for 10 Jan. 2022 but is not carried out until 12 Jan. 2022, and/or can relate to at least one individual aspect of an event, for example a heat disinfection with 85° C. was planned on test point A for 10 Jan. 2022, but a heat disinfection with 79° C. was carried out on test point A on 10 Jan. 2022.

A device according to the invention preferably captures the regulatory documentation provisions applicable at a system location and generates preferably independently a documentation report corresponding to said provisions.

According to an embodiment, the controlling unit is programmed to document, in an automated manner, data regarding at least one sampling and/or disinfection of the system, or the at least one part thereof, which has actually taken place, data regarding at least one associated planned sampling and/or disinfection of the system or the at least one part thereof, data regarding at least one feedback of a user and/or regarding a confirmation from a user and data relating to a personal identification of the user, an identification of the system or the part thereof, a location of the system or the part thereof and/or a sampling and/or an examination of a sample and/or a time identification, preferably in a digital log book.

Another aspect of the present invention relates to a method for interactively carrying out sampling and/or disinfection of a system, more particularly a water technology system, or at least one part thereof, wherein the method is preferably carried out using a device according to the invention, said method having the following steps:
identifying the system or the at least one part thereof,
loading, from a database, historical data relating to least one operating process of the system or the at least one part thereof, and
automatically creating an execution sequence for future sampling and/or disinfection of the system or the part thereof on the basis of the historical data, wherein the execution sequence contains at least one specific task for a user, which is communicated to the user at at least one predefined point in time, wherein
the user is requested, by means of the user interface, to input a feedback regarding the at least one specific task and/or the completion thereof by the user, following which
the execution sequence for sampling and/or disinfection is independently adjusted and/or updated on the basis of the feedback of the user.

Alternatively or in addition to the feedback by the user, a feedback regarding the at least one specific task and/or the completion thereof by the user can be provided by the system and/or the at least one part thereof, for example a reverse osmosis system.

According to an embodiment, a method according to the invention comprises the following step: prompting a user, by means of the user interface, to confirm a created execution sequence for future sampling and/or disinfection of the system or the part thereof.

After confirmation has been provided, an output unit can be triggered to output at least one label, on which preferably a sampling place, a system, or a part thereof, to be sampled, a type of sample, a time of sample-taking, an instruction to examine the sample and/or a name of the person carrying out the sampling is specified. Alternatively or additionally, a dispatch note and/or an executional excerpt can also be output, as described above in the context of a device according to the invention.

In principle, all the features disclosed above with reference to a device according to the invention can also be applied to a method according to the invention, and in the context be read to said method, even if these are not, in order to avoid redundancies, explicitly repeated.

According to an embodiment, a method according to the invention comprises the following step: capturing a location of the system or the at least part thereof, loading the local circumstances currently applicable at this location, more particularly regulatory requirements, public holidays, responsibilities, specified processing intervals and/or operating processes and taking account of the local circumstances during creation and/or adjustment of the execution sequence for future sampling and/or disinfection of the system or the part thereof.

According to an embodiment, a method according to the invention comprises the following step: automatically creating at least one dynamic selection list on the basis of the identified system or the part thereof and preferably an associated location, which selection list preferably enables the user to make a selection regarding the system, at least one part thereof, a planned disinfection method, a planned sampling, a place, a type of planned sampling and/or an examination of samples, wherein the parts of the selection list are automatically adjusted in response to input made by the user by means of the user interface.

According to an embodiment, a method according to the invention comprises the following step: prompting the user, by means of the user interface, to confirm a created execution sequence for future sampling and/or disinfection of the system or the part thereof, for which by means of the user interface the created execution sequence is output in the form of an overview, which enables the user to confirm the execution sequence to be confirmed in a centralized manner with just one action or to reject the confirmation.

According to an embodiment, a method according to the invention comprises the following step: creating an execution sequence for future sampling and disinfection of the system or the part thereof, wherein after each disinfection, sampling is planned, by means of which sampling the effectiveness of the disinfection is validated.

According to an embodiment, the user is provided, by means of the interface, with an option of editing a created execution sequence.

According to an embodiment, a method according to the invention comprises the following steps:
capturing data regarding at least one sampling and/or disinfection of the system, or the at least one part thereof, which has actually taken place,
comparing said data with data regarding at least one associated planned sampling and/or disinfection of the system or the at least one part thereof,
displaying the result of the comparison to the user by means of an output, and
independently adapting and/or updating the created execution sequence for future sampling and/or disinfection on the basis of the captured data and/or of the result of the comparison.

In principle, a created execution sequence can relate not only a future sampling and/or disinfection,
and can also contain, as part of an overview of the past, information relating to sampling
and/or disinfection which is planned or has actually taken place.

According to an embodiment, in a method according to the invention, an automatic assignment mechanism is used which is programmed to capture captured data regarding at least one sampling and/or disinfection of the system, or the at least one part thereof, which has actually taken place and to assign said data to associated data regarding at least one corresponding planned sampling and/or disinfection of the system or the at least one part thereof, and a plausibility check is preferably carried out before the assignment. In other words, the events planned in an execution sequence are preferably compared with events which have actually occurred.

According to an embodiment, data regarding at least one sampling and/or disinfection of the system, or the at least one part thereof, which has actually taken place, data regarding at least one associated planned sampling and/or disinfection of the system or the at least one part thereof, data regarding at least one feedback of a user and/or regarding a confirmation from a user and data relating to a personal identification of the user, an identification of the system or the part thereof, a location of the system or the part thereof and/or a sampling and/or an examination of a sample and/or a time identifier, are documented in an automated manner, preferably in a digital log book.

Preferably, the regulatory provisions currently applicable at a captured location of a system are automatically used, and therefore it is automatically ensured that the documentation corresponds to the applicable provisions.

At this juncture, it is noted that all features disclosed within the framework of a device according to the invention can preferably also be applied to a method according to the invention, and vice versa, although in order to avoid redundancies, these features may not be explicitly mentioned.

A further aspect of the invention relates to a computer program product which contains machine-readable instructions which enable a device to carry out a method according to the invention.

Further features, effects and advantages of the present application result from the following description of embodiments of the invention with reference to the Figures.

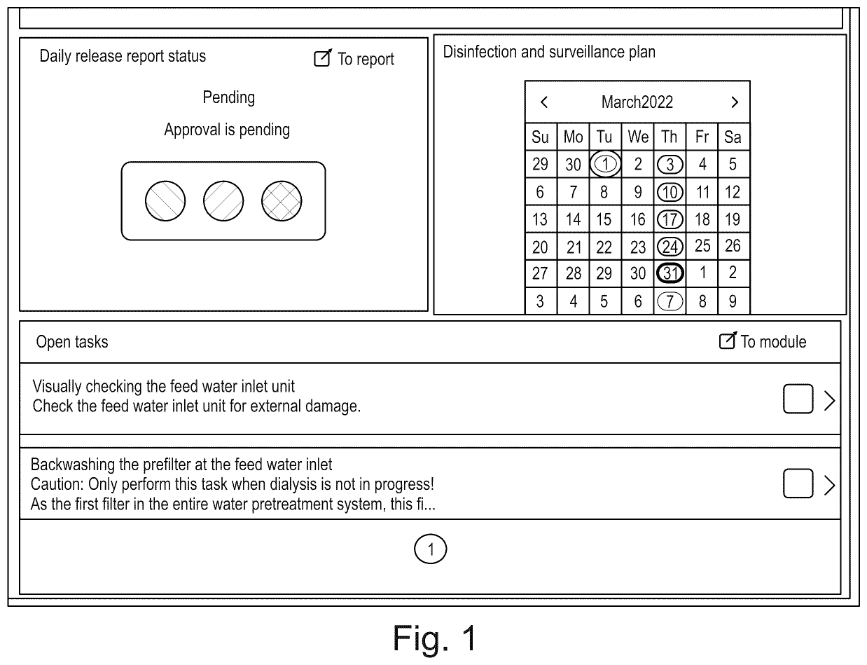

FIG. 1 shows an exemplary view of an overview, output by means of a user interface, of planned disinfections and samplings of a system and specific tasks for the user;

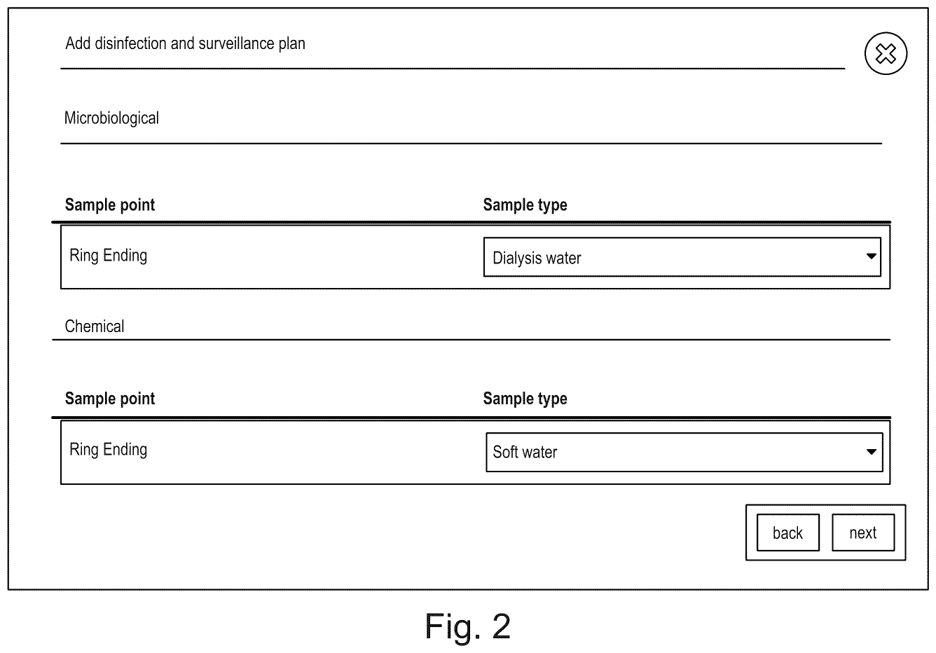

FIG. 2 shows an exemplary view by means of which a user can select, by means of a user interface, a type of sample for a microbiological examination of a sample and a chemical examination of a sample, in each case from a dynamic list;

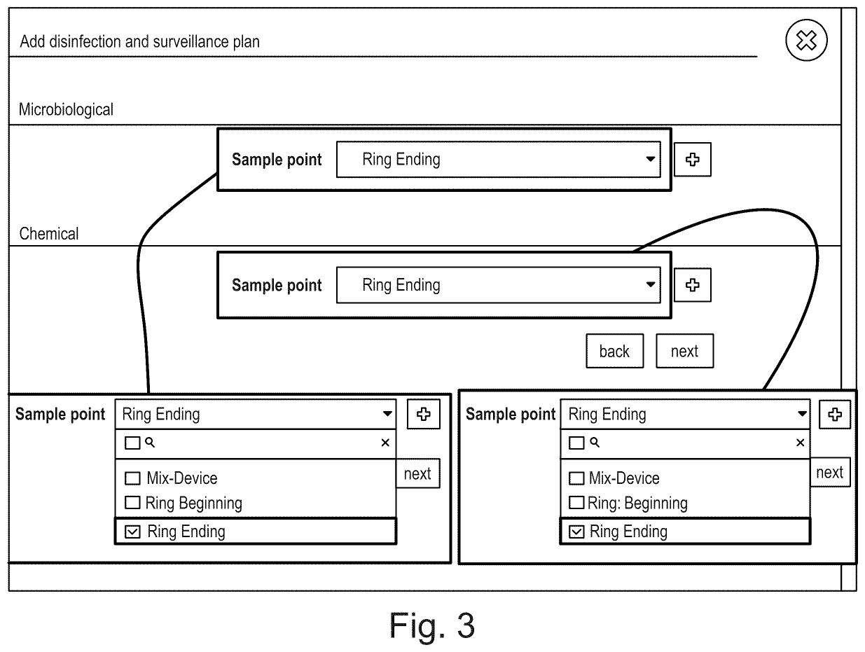

FIG. 3 shows an exemplary view by means of which a user can select, by means of a user interface, a place of sample withdrawal for a microbiological examination of a sample and a chemical examination of a sample, in each case from a dynamic list; and

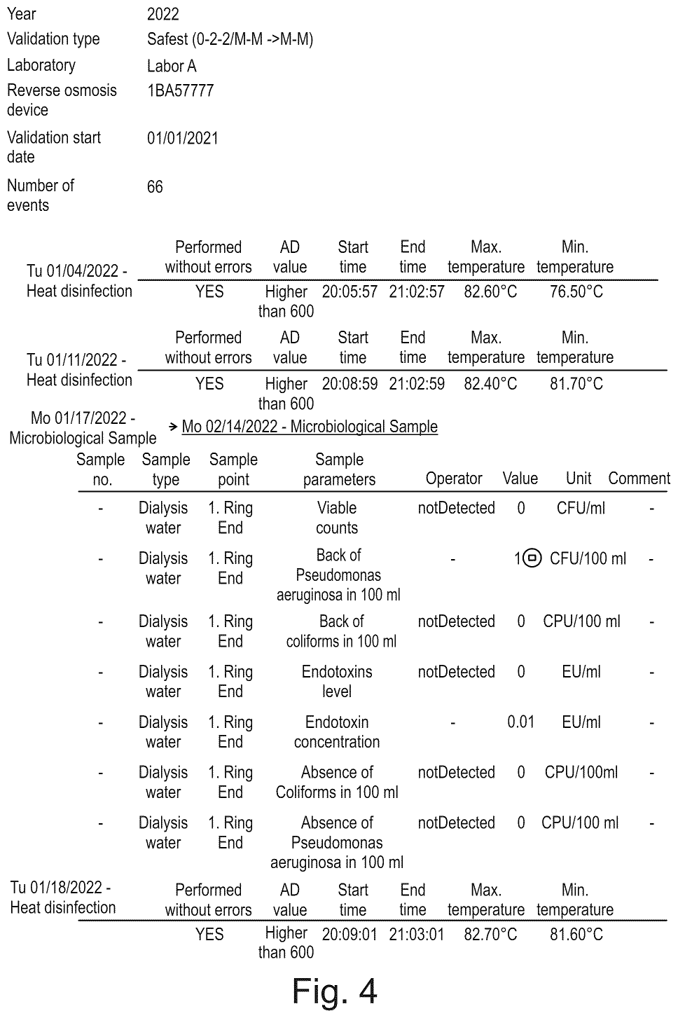

FIG. 4 shows an exemplary view of an automated documentation in which a device according to the invention merges planned events with events that have actually occurred.

From the exemplary view of an overview output by means of a user interface shown in FIG. 1, it can be seen for example that a disinfection of a system is planned for Tuesday 1 Mar. 2022, following which planned sampling is planned on Thursday 3 Mar. 2022.

By means of the graphical user interface (GUI) shown in FIG. 1, the user can for example select the disinfection planned for Tuesday 1 Mar. 2022. A ring is displayed around the date on the calendar view to illustrate the user's selection.

After selection, the user's tasks relating to the selected event are displayed to the user. For example, the user's first task is to visually check the valve of the feed water inlet (“Visually checking the feed water inlet”). The second task consists in backwashing the prefilter at the feed water inlet (“Backwashing the prefilter at the feed water inlet”). If the user clicks on the button for the task, additional information for the task, for example regarding the location of the component to be checked, is displayed to the user.

Furthermore, the user can mark individual tasks as complete, for example, by means of the task buttons.

FIG. 2 shows an exemplary view by means of which a user can select, by means of a user interface, a place of sample-taking, for example a place at the end of a ring line, and a type of sample, for example dialysis water or soft water, for a microbiological examination of a sample and a chemical examination of a sample, in each case from a dynamic list. The lists or selection lists have the form of dropdown menus the parts of which are independently updated and/or preselected by a device.

Preferably, a sample is always taken at the end of a ring line. A ring line refers preferably to a line which is connected to a system for providing medical water and which serves to feed water to at least one, preferably a plurality of treatment apparatuses, more particularly dialysis machines, in a circular manner. Additionally, a sample can also be taken at the start of the ring line and/or any other position. Preferably, the system and the associated line systems are equipped with defined valves for withdrawing samples.

FIG. 3 shows an exemplary view by means of which a user can select, by means of a user interface, a place of sample withdrawal for a microbiological examination of a sample and a chemical examination of a sample, in each case from a dynamic list. In this example, based on the type and location of the system, a dynamic list relating to the place of sample-taking is created, which shows the parts: mixing device, ring start and ring end. In other words, sample-taking can be selected for a mixing device/metering unit, at the start of a ring line or at the end of the ring line. Other places for sample-taking which are not relevant in this example, for example withdrawal on a water feed line to the system, are not contained in the dynamic list for this example.

FIG. 4 shows an exemplary view of an automated documentation in which a device according to the invention merges planned events with events that have actually occurred. The report contains a time identifier and an identification of the carried-out validation or examination and an identification of the system.

As can be seen from the report in FIG. 4, heat disinfections of the system were carried out on 4 Jan. 2022, on 11 Jan. 2022 and on 18 Jan. 2022, during which no errors were captured. The start and end times and the captured maximum and minimum temperatures are documented in the report.

A microbiological examination of a sample of dialysis water planned for 17 Jan. 2022 was carried out not as planned, but instead on 14 Feb. 2022. An arrow in the report shows that the date of the examination was postponed to the underlined date. The user can thus see at a glance the original plan and the event that deviated from the plan.

Furthermore, the user's attention is drawn, by means of a flag icon, to an unexpected value of a colony-forming unit (CFU) per 100 ml of the concentration of Pseudomonas aeruginosa. In this way, the user can see critical measurement values at a glance.

If the user clicks on the flag icon, for example, additional information relating to the examination of Pseudomonas aeruginosa and/or optionally further-reaching measures, such as a replacement of the filter and/or a further examination, are displayed.

Furthermore, a person skilled in the art understands that if an element is specified in the singular with the article “a”, this is understood not in the sense of “exactly one” but in the sense of “at least one”, wherein the disclosure is not restricted to exactly one of these elements.

Furthermore, it is obvious to a person skilled in the art that if certain elements or features of the invention are disclosed in a particular combination or in connection with a particular embodiment, said elements or features of the invention can also be claimed in isolation or in other combinations or in connection with another embodiment.

All features disclosed in conjunction with the method according to the invention can relate equally to a device according to the invention, and vice versa, even if, for the sake of brevity, not all of these features or combinations thereof are expressly disclosed.

1. A device for interactively carrying out sampling and/or disinfection of a system, or at least one part thereof, wherein the device has a user interface and a controlling unit which is programmed
to identify the system or the at least one part thereof,
to read, from a database, historical data relating to at least one operating process of the system of the at least one part thereof or to load same to a memory, and
on the basis of the historical data, to automatically create an execution sequence for future sampling and/or disinfection of the system or the part thereof, wherein the execution sequence contains at least one specific task for a user, which is communicated to the user at at least one predefined point in time, wherein
the user is requested, by means of the user interface, to input a feedback regarding the at least one specific task and/or the completion thereof, and/or
the controlling unit obtains a feedback regarding the at least one specific task and/or the completion thereof by the user from the system or the at least one part thereof, following which
the controlling unit independently adjusts and/or updates the execution sequence for sampling and/or disinfection on the basis of the feedback or feedbacks.

2. The device according to claim 1, wherein the controlling unit is further programmed to prompt a user, by means of the user interface, to confirm a created execution sequence for future sampling and/or disinfection of the system or the part thereof and, after confirmation has been provided, to trigger an output unit to output at least one label, on which a place of sampling, a system, or a part thereof, to be sampled, a type of sample, a time of sample-taking, an instruction to examine the sample and/or a name of the person carrying out sampling is specified, a dispatch note and/or a executional excerpt or an action instruction.

3. The device according to claim 1, wherein the controlling unit is further programmed to capture a location of the system or the at least one part thereof, to read the local circumstances currently applicable at this location, or to load same to a memory, and to take account of the local circumstances during creation of the execution sequence for future sampling and/or disinfection of the system or the part thereof.

4. The device according to claim 1, wherein the controlling unit is further programmed to automatically create at least one dynamic selection list on the basis of the identified system or the part thereof and preferably an associated location, which selection list enables the user to make a selection regarding the system, at least one part thereof, a planned disinfection method, a planned sampling, a place of sample-taking, a type of planned sampling and/or an examination of samples, wherein the parts of the selection list are automatically adjusted in response to input made by the user by means of the user interface.

5. The device according to claim 1, wherein the controlling unit is further programmed to prompt a user, by means of the user interface, to confirm a created execution sequence for future sampling and/or disinfection of the system or the part thereof, for which by means of the user interface the created execution sequence is output in the form of an overview, which enables the user to confirm the execution sequence to be confirmed in a centralized manner with just one action or to reject the confirmation.

6. The device according to claim 1, wherein the controlling unit is further programmed to create an execution sequence for future sampling and disinfection of the system or the part thereof, wherein the times of disinfection and sampling are considered automatically in a coordinated manner and each sampling is preceded by a disinfection, and therefore the effectiveness of the disinfection is validated by the sampling.

7. The device according to claim 1, wherein the controlling unit is further programmed to provide the user, by means of the interface, with an option of editing a created execution sequence.

8. The device according to claim 1, wherein the controlling unit is further programmed to capture digital data regarding at least one sampling and/or disinfection of the system, or the at least one part thereof, which has actually taken place and to compare same with data regarding at least one associated planned sampling and/or disinfection of the system or the at least one part thereof, and to display the result of the comparison to the user by means of an output and/or to independently adjust and/or update the created execution sequence for future sampling and/or disinfection on the basis of the captured data and/or the result of the comparison.

9. The device according to claim 8, wherein the controlling unit further has an automatic assignment mechanism which is programmed to capture captured data regarding at least one sampling and/or disinfection of the system, or the at least one part thereof, which has actually taken place and to assign said data to associated data regarding at least one sampling and/or disinfection of the system, or the at least one part thereof, planned as part of the execution sequence and the controlling unit carries out a plausibility check before the assignment.

10. The device according to claim 1, wherein the controlling unit is further programmed to document, in an automated manner, data regarding at least one sampling and/or disinfection of the system, or the at least one part thereof, which has actually taken place, data regarding at least one associated sampling and/or disinfection of the system, or the at least one part thereof, planned as part of the execution sequence, data regarding at least one feedback of a user and/or a feedback by the system or a part thereof and/or regarding a confirmation from a user and data relating to a personal identification of the user, an identification of the system or the part thereof, a location of the system or the part thereof and/or a sampling and/or an examination of a sample and/or a time identifier.

11. A computer-implemented method for interactively carrying out sampling and/or disinfection of a system, or at least one part thereof, wherein the method is carried out using the device according to claim 1, said method having the following steps:
identifying the system or the at least one part thereof,
reading, from a database, historical data relating to least one operating process of the system or the at least one part thereof and/or loading said historical data to a memory, and
automatically creating, by the controlling unit, an execution sequence for future sampling and/or disinfection of the system or the part thereof on the basis of the historical data, wherein the execution sequence contains at least one specific task for a user, which is communicated to the user at at least one predefined point in time, wherein
the user is requested, by means of the user interface, to input a feedback regarding the at least one specific task and/or the completion thereof, and/or the controlling unit obtains a feedback regarding the at least one specific task and/or the completion thereof by the user from the system or the at least one part thereof, following which
the execution sequence for sampling and/or disinfection is independently adjusted and/or updated by the controlling unit on the basis of the feedback or feedbacks.

12. The method according to claim 11, further comprising the step of: prompting a user, by means of the user interface, to confirm a created execution sequence for future sampling and/or disinfection of the system or the part thereof and, after confirmation has been provided, causing an output unit to output at least one label, on which a place of sampling, a system, or a part thereof, to be sampled, a type of sample, a time of sample-taking, an instruction to examine the sample and/or a name of the person carrying out sampling is specified, a dispatch note or a executional excerpt or an action instruction.

13. The method according to claim 11, further comprising the step of: capturing a location of the system or the at least part thereof, reading or loading from a database the local circumstances currently applicable at this location and taking account of the local circumstances during creation and/or adjustment of the execution sequence for future sampling and/or disinfection of the system or the part thereof.

14. The method according to claim 11, further comprising the step of: automatically creating at least one dynamic selection list on the basis of the identified system or the part thereof and an associated location, which selection list enables the user to make a selection regarding the system, at least one part thereof, a planned disinfection method, a planned sampling, a place, a type of planned sampling and/or an examination of samples, wherein the parts of the selection list are automatically adjusted in response to input made by the user by means of the user interface.

15. The method according to claim 11, further comprising the step of: prompting the user, by means of the user interface, to confirm a created execution sequence for future sampling and/or disinfection of the system or the part thereof, for which by means of the user interface the created execution sequence is output in the form of an overview, which enables the user to confirm the execution sequence to be confirmed in a centralized manner with just one action or to reject the confirmation.

16. The method according to claim 11, further comprising the step of: creating an execution sequence for future sampling and disinfection of the system or the part thereof, wherein the times of disinfection and sampling are considered automatically in a coordinated manner and each sampling is preceded by a disinfection, and therefore the effectiveness of the disinfection is validated by the sampling.

17. The method according to claim 11, wherein the user is provided, by means of the interface, with an option of editing a created execution sequence.

18. The method according to claim 11, further comprising the step of:
capturing data regarding at least one sampling and/or disinfection of the system, or the at least one part thereof, which has actually taken place,
comparing said data with data regarding at least one associated sampling and/or disinfection of the system, or the at least one part thereof, planned as part of the execution sequence,
displaying the result of the comparison to the user by means of an output unit, and
independently adapting and/or updating, by means of the controlling unit, the created execution sequence for future sampling and/or disinfection on the basis of the captured data and/or of the result of the comparison.

19. The method according to claim 18, wherein an automatic assignment mechanism is used, which is programmed to capture captured data regarding at least one sampling and/or disinfection of the system, or the at least one part thereof, which has actually taken place and to assign said data to associated data regarding at least one sampling and/or disinfection of the system, or the at least one part thereof, planned as part of the execution sequence and the controlling unit carries out a plausibility check before the assignment.

20. The method according to claim 11, wherein data regarding at least one sampling and/or disinfection of the system, or the at least one part thereof, which has actually taken place, data regarding at least one associated sampling and/or disinfection of the system, or the at least one part thereof, planned as part of the execution sequence, data regarding at least one feedback of a user and/or of the system or a part thereof and/or regarding a confirmation and/or editing of the execution sequence by a user and data relating to a personal identification of the user, an identification of the system or the part thereof, a location of the system or the part thereof and/or a sampling and/or an examination of a sample and/or a time identifier, are automatically documented.

21. A computer program product which contains machine-readable instructions which enable a device to carry out a method according to claim 11.
